(19) United States
(12) Patent Application Publication
(10) Pub. No.: US 2021/0263042 A1
(43) Pub. Date: Aug. 26, 2021

(54) TANDEM-PAIRED COLUMN CHEMISTRY FOR HIGH-THROUGHPUT PROTEOMIC EXOSOME ANALYSIS

(71) Applicant: NX Prenatal Inc., Louisville, KY (US)

(72) Inventors: Kevin P. ROSENBLATT, Louisville, KY (US); Najmuddin KHAJA, Louisville, KY (US); Malik KHALID, Louisville, KY (US)

(21) Appl. No.: 17/314,665

(22) Filed: May 7, 2021

Related U.S. Application Data
(63) Continuation of application No. PCT/US2019/060660, filed on Nov. 9, 2019.
(60) Provisional application No. 62/757,922, filed on Nov. 9, 2018.

(51) Int. Cl. G01N 33/68; B01D 15/32; B01D 15/34; G01N 1/40; G01N 30/08; G01N 30/88

(52) U.S. Cl. CPC G01N 33/6848; B01D 15/325; B01D 15/34; G01N 1/4044; G01N 1/405; G01N 2030/8831; G01N 30/08; G01N 30/88; G01N 2570/00; G01N 2030/085; G01N 33/689

(57) ABSTRACT

Compositions and methods for sample preparation and mass spectrometric analysis of peptide samples obtained from biological samples are provided. The compositions and methods include a tandem column system in which a trap column is in fluid contact with an analytical column such as, for example, a HPLC column. As analytes are eluted from the analytical column, they can be passed to a detector (e.g., a mass spectrometer) for peptide analysis.

### REFERENCE TO RELATED APPLICATIONS

This application claims the benefit of the priority date of U.S. application 62/757,922 filed Nov. 9, 2018 (Rosenblatt et al.), the contents of which are incorporated herein in their entirety.

### STATEMENT AS TO FEDERALLY SPONSORED RESEARCH

None.

### THE NAMES OF THE PARTIES TO A JOINT RESEARCH AGREEMENT

None.

### SEQUENCE LISTING

None.

### TECHNICAL FIELD

This disclosure is in the field of sample preparation for peptide analysis, e.g., analysis by high performance liquid chromatography and/or mass spectrometry.

### BACKGROUND

Many diagnostic procedures rely on proteomic analyses. The proteome, i.e., the unique collection of proteins present in a particular cell at a particular developmental state, can provide a wealth of information about the condition of the cell, and has found use in diagnosis and treatment strategies for, among other conditions, cancers and disorders of pregnancy such as spontaneous pre-term birth (SPTB) and preeclampsia.

High performance liquid chromatography (HPLC) is used frequently in the analysis of biological samples, due to its rapidity and high resolving power. For cellular samples, extracellular vesicles, also known as circulating microparticles (e.g., exosomes) are often a convenient source of material for analysis. Exosomes contain protein, lipids, nucleic acids, carbohydrates and ions; so, for proteomic analyses, it would be desirable to remove non-peptide materials from exosome samples. This would prevent overloading and poor performance of an analytical column (such as a HPLC column) and facilitate subsequent analyses, e.g., by mass spectrometry. In addition, it would minimize the possibility that the column chemistry will be altered by interaction with the sample.

Most of the current methods for proteomic analysis involve disruption of a cellular sample using, e.g., denaturants or chaotropic agents; followed by one or more purification or enrichment steps that remove non-protein contaminants and enrich for protein or peptide constituents; followed by proteolysis of the enriched protein sample to generate a collection of peptides; followed by analysis of the peptides, for example, by mass spectrometry (MS). See, for example, Anderson & Hunter (2006) Mol. Cell. Proteomics 5(4):573-588.

Three major techniques for enriching cellular samples for protein, prior to MS analysis, are (1) chromatography, e.g., HPLC chromatography using, e.g., a Sep Pak (Waters, Milford, MA) or a ZipTip (Millipore, Billerica, MA), (2) gel electrophoresis and (3) organic extraction and precipitation. After the protein enrichment step, samples are subjected to proteolysis, and the peptide fragments are analyzed by MS.

There are a number of problems associated with these current methods for proteomic analysis. First, cellular samples contain high concentrations of macromolecules other than proteins, such as lipids, carbohydrates and nucleic acids, and salts, such as sodium chloride (NaCl). These can interfere with subsequent processing steps (such as, for example, proteolysis), can overload analytical instruments (such as chromatography columns), and can cause high background signals in mass spectrometric analyses. In addition to overloading columns, non-protein macromolecules and salts can alter the chemistry of HPLC columns (e.g., by causing alterations in pH and ionic strength along the column); thereby altering the interaction of peptides with the column matrix, and leading to irreproducibility in the analysis of samples from different sources. Even though current methods often contain a step in which the sample is enriched for proteins and peptides, current enrichment methods are not always sufficient to provide material pure enough for robust MS analysis, particularly with respect to the large sample sizes required for optimum sensitivity, and sample loss during those purification steps can change the quantification of peptides in a sample.

A second problem with current methods is the difficulty of obtaining, from a biological sample such as a blood draw, sufficient material for MS analysis.

A third problem with current methods is that, because of the small fluid volumes (and hence low flow rates required to achieve optimal sensitivity) involved, they are time-consuming.

A fourth problem with current methods is that sample preparation techniques often result in the generation of a peptide solution having a high ionic strength, which affects the interaction of peptides with HPLC resins and reduces the sensitivity of MS analysis.

A fifth problem with current methods is that they comprise a series of multiple steps, with sample loss occurring at each step.

Another approach to increasing sensitivity is to obtain samples from extracellular vesicles (e.g., exosomes) rather than from cells themselves. However, even using samples obtained from extracellular vesicles, problems with purity and sensitivity remain.

In summary, current methods for proteomic analysis suffer, for the reasons set forth above, from inadequate sensitivity and inaccuracies in target peptide/protein quantification. In addition, current methods are time-consuming and have poor yield. Accordingly, improved methods and systems for analysis of biological samples are needed.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. The invention will be more particularly described in conjunction with the following drawings wherein:

FIG. 1 shows an exemplary tandem column system 1 comprising a trap column 10 in fluid contact with an analytical column 20. via fluid line 15 dispersed between outlet 11 of trap column 10 and inlet 21 of analytical column 20.

FIG. 2 show another exemplary tandem column system 2 comprising trap column 10, multiport valve 30 and analytical column 20. Trap column 10 is in fluid communication with valve 30 via first fluid conduit 40 which attaches to outlet 11 of trap column 10 and to intake port 32 on valve 30. Valve 30 is, in turn, in fluid communication with analytical column 20 via second fluid conduit 50 attached to outlet port 31 of valve 30 and inlet 21 of analytical column 20. The system optionally comprises third fluid conduit 70 attached to outlet port 33 of valve 30 which is optionally attached, or flows into, waste container 60.

FIG. 3 show another exemplary tandem column system 3 comprising trap column 10, multiport valve 30, analytical column 20 and detector 80. Trap column 10 is in fluid communication with valve 30 via fluid conduit 40 which attaches to outlet 11 of trap column 10 and to intake port 32 on valve 30. Valve 30 is, in turn, in fluid communication with analytical column 20 via fluid conduit 50 attached to outlet port 31 of valve 30 and inlet 21 of analytical column 20. Analytical column 20 is in fluid contact with detector 80 via fourth fluid conduit 90 attached to analytical column outlet 22 and detector inlet 81. The system optionally comprises third fluid conduit 70 attached to outlet port 33 of valve 30 which is optionally attached, or flows into, waste container 60.

FIG. 4 shows another exemplary tandem column system 4 comprising trap column 10, multiport valve 30, analytical column 20, detector 80 and pump 100. Pump 100 is in fluid communication with trap column 10 via fifth fluid conduit 110 attached to pump outlet 101 and trap column inlet 11. Trap column 10 is in fluid communication with valve 30 via fluid conduit 40 which attaches to outlet 11 of trap column 10 and to intake port 32 on valve 30. Valve 30 is, in turn, in fluid communication with analytical column 20 via fluid conduit 50 attached to outlet port 31 of valve 30 and inlet 21 of analytical column 20. Analytical column 20 is in fluid contact with detector 80 via fourth fluid conduit 90 attached to analytical column outlet 22 and detector inlet 81. The system optionally comprises third fluid conduit 70 attached to outlet port 33 of valve 30 which is optionally attached, or flows into, waste container 60.

FIG. 5 shows another exemplary tandem column system. An analytical pump pumps sample through an autosampler into a trap column. The trap column is connected to a multiport valve which has ports (referred to as “Viper plugs”) leading to Waste, a Loading Pump and an Analytical column. The loading pump pumps sample eluted from the trap column into the analytical column. The Analytical column, in turn, is connected to a mass spectrometer.

FIGS. 6A and 6B show mass spectrometry results of an ITIH4 peptide from exosome enriched samples disrupted with urea and digested with trypsin. In FIG. 6A the sample is loaded directly onto the analytical column before mass spectrometry analysis. In FIG. 6B, the sample is first passed through a trap column before mass spectrometry analysis.

FIGS. 7A and 7B show mass spectrometry results of an 101 peptide from exosome enriched samples disrupted with urea and digested with trypsin. In FIG. 7A the sample is loaded directly onto the analytical column before mass spectrometry analysis. In FIG. 7B, the sample is first passed through a trap column before mass spectrometry analysis.

FIG. 8 shows various microparticles produced by a cell, including exosomes, microvesicles, and apoptotic bodies.

### SUMMARY

Disclosed herein are methods and compositions for preparing and analyzing biological samples.

In certain embodiments, the sample is a biological fluid, such as, for example, blood, serum, plasma, saliva, cerebrospinal fluid, tears, amniotic fluid or urine. In additional embodiments, the sample is a tissue sample, for example, a biopsy or a buccal swab. The sample can be obtained from a subject who has, or is suspected of having, or who is being screened for, a pathological condition. Exemplary pathological conditions are described elsewhere herein but can include cancers, diabetes and disorders of pregnancy such as spontaneous preterm birth (SPTB) and preeclampsia.

In certain embodiments, the biological sample is enriched for microparticles such as, for example, exosomes. Enrichment for microparticles can be achieved, for example, by one or more of size exclusion chromatography, ultrafiltration or reverse-phase chromatography.

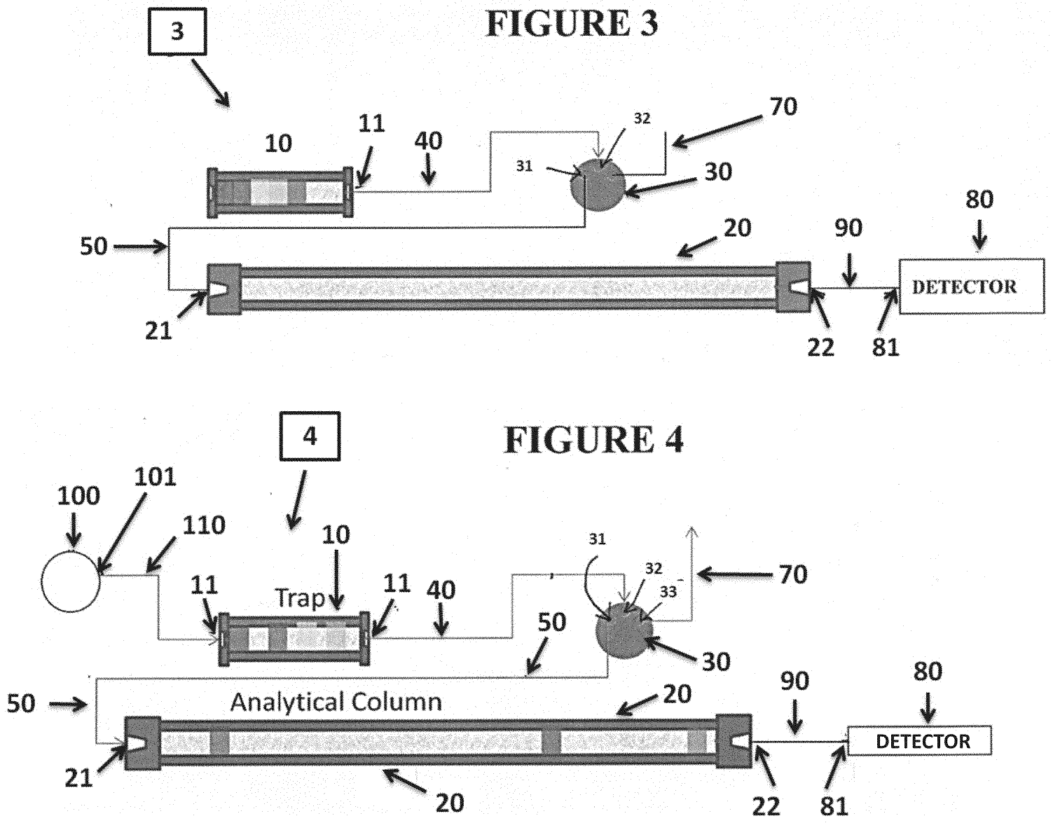

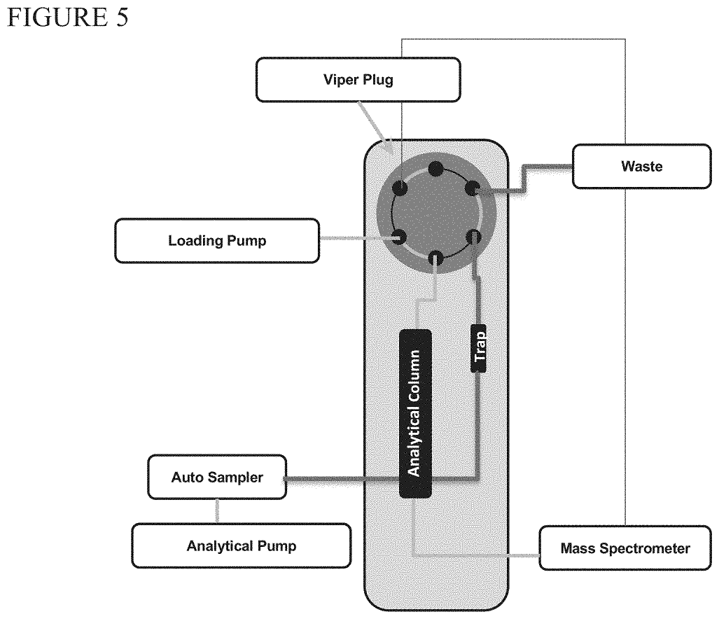

In certain embodiments, a composition as disclosed herein is a tandem paired column system 1 for enriching a biological sample for peptides and for separating the enriched peptides. The system comprises a trap column 10 and an analytical column (or “separation column”) 20 in fluid contact with each other, e.g., through one or more fluid conduit(s) or fluid line(s). Optionally, a multi-port (e.g., 2, 3, 4, 5, 6 or more ports) valve 30 is disposed between the trap column 10 and the analytical column 20 (FIG. 2). In one embodiment, the valve has three ports: an intake port 31 from the trap column, an outlet port 32 to the analytical column, and an outlet port 33 to waste. In certain embodiments, the analytical column 20 is in fluid contact with a detector 80 (e.g., a mass spectrometer) (FIG. 3). In additional embodiments, the system is in contact with a pump 100, optionally upstream and in fluid connection with the trap column 10 (FIG. 4).

In additional embodiments, the system comprises (a) a trap column 10, (b) a multi-port valve 30 connected through a first fluid conduit 40 to the trap column 10, (c) an analytical column 20 connected through a second fluid conduit 50 to the multiport valve 30, and (d) a third fluid conduit 70 connected to the multiport valve 30; wherein the trap column 10 is loaded with a sample enriched for circulating microparticles (e.g., exosomes), wherein the sample has been subjected to particle disruption and proteolytic digestion.

In certain embodiments, either the sample or the trap column 10 comprises stable isotope standard (SIS) peptides.

In additional embodiments, the system further comprises a detector 80 connected through a fourth fluid conduit 90 to the analytical column 20. In additional embodiments, the system further comprises a pump 100 connected to the trap column 10 through a fifth fluid conduit 110. Detector 80 and pump 100 can both be present in the system, or one or the other can be present.

In the systems described herein, trap column 10 can be a high performance liquid chromatography (HPLC) column (e.g., a C18 column or a C6 column), a reverse phase chromatography (RPC) column, an anion exchange column, a cation exchange column or a size exclusion column.

In the systems described herein, analytical column 20 can be a high performance liquid chromatography (HPLC) column (e.g., a C18 column or a C6 column), a reverse phase chromatography (RPC) column, an anion exchange column, a cation exchange column or a size exclusion column.

In certain embodiments, the systems disclosed herein comprise a detector 80 for identifying and quantitating peptides eluted from analytical column 20. The detector can be a mass spectrometer, for example, a quadrupole mass spectrometer, a time-of-flight mass spectrometer, a tandem mass spectrometer, such as a triple quadrupole mass spectrometer. In certain embodiments, the detector comprises a triple quadrupole mass spectrometer and detection comprises multiple reaction monitoring.

In certain embodiments for the use of the system described in the previous paragraph, the trap column 10 is loaded with a biological sample enriched for circulating microparticles (e.g., exosomes), wherein the sample has been subjected to particle disruption and proteolysis. Disruption of microparticles is achieved by, for example, treatment of the microparticles with one or more of a denaturing agent (e.g., urea, guanidinium chloride, sodium perchlorate, lithium perchlorate, lithium acetate or thiourea); a reducing agent such as, for example, dithiothreitol (DTT), dithioerythritol (DTE), tris (2-carboxyethyl) phosphine, glutathione (e.g., L-glutathione) or β-mercaptoethanol; and/or an alkylating agent such as, for example, iodoacetamide or iodoacetate (which bind to the thiol group of cysteine residues, thereby breaking disulfide bonds). Other disulfide bond-breaking agents, as are known in the art, can also be used in place of, or together with, an alkylating agent as disclosed herein.

Proteolytic enzymes and proteolytic agents are well-known in the art. Proteolysis of disrupted microparticles can be achieved by exposure of disrupted microparticles (e.g., exosomes) to an aminopeptidase, a carboxypeptidase, or any one or more of the enzymes trypsin, chymotrypsin, bromelian, papain, pronase or Proteinase K. The chemical agent cyanogen bromide (CnBr) can also be used as a proteolytic agent.

The microparticle sample (e.g., exosome sample) that has been subjected to particle disruption and proteolysis can also include one or more peptide targets (standards) (i.e., a peptide of known sequence), to assist in detection of peptides in the sample. In certain embodiments, a peptide standard labeled with one or more heavy isotopes is used to generate a stable-isotope standard (SIS) peptide for the quantification of and verification of the presence of a target peptide.

The trap column 10 onto which the microparticle sample (e.g., exosome sample) that has been subjected to particle disruption and proteolysis can be loaded, for example, onto a reverse phase column, an anion exchange column, a cation exchange column, a polystyrene-divinlybenzene column or a size exclusion column. The trap column 10 can also be a high-performance liquid chromatography (HPLC) column using a reverse phase resin such as, for example, C18 or C6.

In addition to proteins, preparations of circulating microparticles such as exosomes contain lipids, nucleic acids, carbohydrates, small metabolic molecules, and salts. In the methods disclosed herein, these non-protein constituents of the microparticles are not retained by the trap column 10: they either flow through or are washed off after application of sample to the trap column. Peptides, produced by proteolysis as described above, are retained by the trap column 10 and can be eluted for separation on an analytical column 20. Accordingly, in certain embodiments uncaptured material comprises salts, small molecules, macromolecules (such as lipids, nucleic acids and carbohydrates) and complexes having a size greater than 1 kD. In additional embodiments, uncaptured material has a size greater than 0.5 kD, or greater than 2 kD, or greater than 3 KD, or greater than 4 kD, or greater than 5 kD. In some embodiments the uncaptured material comprises polypeptides having a length greater than about 50 amino acids or about 100 amino acids.

Peptide material that has bound to, and been eluted from, the trap column 10 is passed to an analytical column 20 for separation, optionally via a valve 30 that allows column effluent to be passed either to waste 60 or to the analytical column 20. The analytical column 20 can be a high performance liquid chromatography (HPLC) column, comprising a reverse phase resin such as C18 or C6. As noted, the trap column 10 can be in fluid connection with the analytical column 20, so that material exiting the trap column 10 is loaded directly onto the analytical column 20, thereby minimizing time and preventing losses of material. In certain embodiments, the fluid connection between the trap column 10 and the analytical column 20 comprises a first fluid conduit 40 in fluid contact with the trap column 10 and with a multiport valve 30 via intake port 31, which valve 30 is in fluid contact, via outlet port 32, with a second fluid conduit 50, which second fluid conduit 50 is in fluid contact with the analytical column 20. That is, the fluid connection between the trap column 10 and the analytical column 20 can comprise a first fluid conduit 40 and a second fluid conduit 50, with a multiport valve 30 disposed therebetween.

Peptide analytes eluted from analytical column 20 are provided to detector 80 for analysis. In certain embodiments, detection comprises providing separated analytes to detector 80 through a fluid conduit 90 between analytical column 20 and detector 80. In certain embodiments, detector 80 detects analytes by mass spectrometry. In some of these embodiments, detector 80 comprises a mass spectrometer such as, for example, a quadrupole mass spectrometer, a time-of-flight mass spectrometer, a triple quadrupole mass spectrometer, or some other type of tandem mass spectrometer. In certain embodiments, the detector 80 comprises a triple quadrupole mass spectrometer and identification and quantification comprises multiple reaction monitoring.

In additional embodiments, provided herein are methods that utilize the systems described herein, wherein the methods comprise (a) loading trap column 10 with a sample comprising disrupted exosomes that have been subjected to a proteolytic treatment such that the flow-through passes through first fluid conduit 40, multi-port valve 30, and third fluid conduit 70; (b) washing trap column 10 such that the wash passes through first fluid conduit 40, multi-port valve 30, and third fluid conduit 70; (c) eluting trap column 10 such that the eluate passes through first fluid conduit 40, multi-port valve 30 and second fluid conduit 50 onto analytical column 20; (d) separating the components of the eluate on analytical column 20; (e) passing the separated components of step (d) through fourth fluid conduit 90 to detector 80; and detecting one or more of the separated components. The detector can be, without limitation, a quadrupole mass spectrometer, a time-of-flight mass spectrometer or a tandem mass spectrometer, such as a triple quadrupole mass spectrometer. Detection by mass spectrometry can be, for example, by multiple reaction monitoring (MRM) mass spectrometry and detection can include the use of SIS peptides. For example, in MRM-MS, the sample is treated with a protease to convert full-length proteins to peptide fragments. If one is testing for a specific protein, the sample is spiked with peptide fragment(s), containing stable heavy isotopes (SIS peptides), that have the same sequence as those expected to be produced from the protein for which one is testing. Detection of both heavy and unlabeled, endogenous versions of the same peptide(s) confirms the presence of the protein for which one is testing.

The methods and compositions for sample preparation and analysis disclosed herein make it possible to obtain biological samples of sufficiently high concentration and purity to enable sensitive peptide analysis by mass spectrometry. In contrast to previous methods, it is possible to use complex mixtures of disrupted microparticles as starting material for the analysis (e.g., disrupted exosomes that have been subjected to proteolytic digestion). Such mixtures can be loaded on the trap column without enrichment for protein and the attendant losses that accompany such enrichment procedures. Previous methods have required a protein enrichment step prior to separation and analysis prior to injecting the sample into the HPLC-mass spectrometry system (see above). In certain embodiments, between 5 and 200 micrograms (or any integral value therebetween) of protein or peptide are loaded onto the trap column. In certain embodiments, 5 μg of protein or peptide are loaded onto the trap column.

Enhanced sensitivity also arises from the fact that exosomal peptides are retained on trap column 10, and only a small portion of the retained peptides are eluted at a time onto analytical column 20. In this way, analytical column 20 is not overloaded, and the analytes can be separated conventionally and eluted into detector 80 (e.g., a mass spectrometer) for, e.g., MRM analysis. The methods also utilize a continuous flow rate matched to the speed of detector 80.

In one aspect provided herein is a method comprising: (a) providing a biological sample; (b) enriching the sample for circulating microparticles; (c) subjecting the enriched sample to particle disruption and proteolytic digestion to produce an analytic sample; (d) loading the analytic sample onto a trap column that captures polypeptides; (e) washing uncaptured material from the trap column; (f) eluting the captured polypeptides from the trap column and loading the eluted polypeptides onto an analytical column that is in fluid connection with the trap column; (g) separating the polypeptides on the analytical column; and (h) detecting separated polypeptides with a detector. In one embodiment the biological sample comprises blood, serum, plasma, saliva, cerebrospinal fluid, amniotic fluid or urine. In another embodiment the biological sample is provided from a subject having a condition selected from pregnancy, cancer, spontaneous pre-term birth, pre-eclampsia or diabetes. In another embodiment the biological sample is enriched for microparticles by one or more of size exclusion chromatography, ultrafiltration or reverse-phase chromatography. In another embodiment the circulating microparticles are exosomes. In another embodiment the particle disruption comprises exposure to one or more of urea, a reducing agent (such as, DTT, DTE, mercaptoethanol, tris (2-carboxyethyl) phosphine or glutathione) or iodoacetamide. In another embodiment proteolytic digestion comprises exposing the sample to cyanogen bromide (CnBr) or to one or more proteases such as an aminopeptidase, a carboxypeptidase, trypsin, chymotrypsin, bromelian, papain, pronase, and proteinase k. In another embodiment the method comprises adding stable isotope standard peptides for detecting specific protein markers to the analytical sample. In another embodiment the trap column performs chromatography selected from reverse phase chromatography, anion exchange chromatography, cation exchange chromatography or size exclusion chromatography or is a polystyrene-divinlybenzene column. In another embodiment the trap column comprises a high performance liquid chromatography (HPLC) column. In another embodiment the HPLC column comprises a reverse phase material selected from C18 or C6. In another embodiment the uncaptured material in step (e) comprises material selected from salts and macromolecules or complexes having a size greater than 5.5 kD or the macromolecule is a polypeptide greater than about 50 amino acids long. In another embodiment the analytical column comprises a high-performance liquid chromatography (HPLC) column. In another embodiment the HPLC column comprises a reverse phase material selected from C18 or C6. In another embodiment the fluid connection between the trap column and the analytical column comprises a first fluid conduit and a second fluid conduit, with a multiport valve disposed therebetween. In another embodiment detecting comprises providing separated analytes to the detector through a fluid connection between the analytical column and the detector. In another embodiment the detector detects analytes by mass spectrometry. In another embodiment wherein the detector comprises a mass spectrometer selected from quadrupole mass spectrometer or time-of-flight mass spectrometer. In another embodiment the detector comprises a tandem mass spectrometer. In another embodiment the detector comprises a triple quadrupole mass spectrometer. In another embodiment the detector comprises a triple quadrupole mass spectrometer and detection comprises multiple reaction monitoring.

In another aspect provided herein is a system comprising: (a) a trap column; (b) a multi-port valve connected through a first fluid conduit to the trap column; and (c) an analytical column connected through a second fluid conduit to the multi-port valve. In another embodiment the trap column is loaded with a sample enriched for circulating microparticles, wherein the sample has been subjected to particle disruption and proteolytic digestion. In another embodiment the trap column further comprises stable isotope standard polypeptides. In another embodiment the system further comprises: an outlet fluid conduit connecting an outlet port to the multiport valve. In another embodiment the system further comprises: a detector connected through a detector fluid conduit to the analytical column. In another embodiment the system further comprises: a pump, wherein the pump is connected to the trap column through a pump fluid conduit. In another embodiment the trap column is selected from a reverse phase chromatography column, an anion exchange chromatography column, a cation exchange chromatography column, a polystyrene-divinylbenzene column or a size exclusion column. In another embodiment the trap column comprises a high-performance liquid chromatography (HPLC) column. In another embodiment the HPLC column comprises a reverse phase material selected from C18 or C6. In another embodiment the analytical column comprises a high-performance liquid chromatography (HPLC) column. In another embodiment the detector comprises a mass spectrometer. In another embodiment the detector is a mass spectrometer selected from quadrupole mass spectrometer or time-of-flight mass spectrometer. In another embodiment the detector comprises a tandem mass spectrometer. In another embodiment the detector comprises a triple quadrupole mass spectrometer. In another embodiment the circulating microparticles are exosomes.

In another aspect provided herein is a method comprising: (a) providing a system comprising: (i) a trap column; (ii) a multi-port valve connected through a first fluid conduit to the trap column; (iii) an analytical column connected through a second fluid conduit to the multi-port valve; (iv) an outlet port connected to the multiport valve through outlet fluid conduit; and (v) a detector connected through a detector fluid conduit to the analytical column; (b) loading the trap column with a sample comprising one or a plurality of molecules selected from polypeptides, complex carbohydrates, nucleic acids and, complex lipids, such that the flow-through passes through the first fluid conduit, the multi-port valve, and a third fluid conduit; (c) washing the trap column such that the wash passes through the first fluid conduit, the multi-port valve, and the third fluid conduit; (d) eluting the trap column such that the eluate passes through the first fluid conduit, the multi-port valve and the second fluid conduit onto the analytical column; (e) separating the components of the eluate on the analytical column; (f) passing the separated components of step (d) through the fourth fluid conduit to the detector; and (g) detecting one or more of the separated components. In another embodiment detecting comprises mass spectrometry. In another embodiment mass spectrometry comprises multiple reaction monitoring mass spectrometry. In another embodiment the sample of step (a) comprises between about 1 μg and about 5 μg of protein or peptide. In another embodiment the sample comprises disrupted exosomes that have been subjected to a proteolytic treatment. In another embodiment the sample comprises a plurality of molecules selected from polypeptides, complex carbohydrates, nucleic acids and, complex lipids. In another embodiment the sample further comprises a salt. In another embodiment the sample comprises a homogenate of cells or a tissue homogenate. In another embodiment the sample comprises polypeptides, lipids and salts. In another embodiment the sample further comprises nucleic acids and/or complex carbohydrates. In another embodiment the sample further comprises metals.

### DETAILED DESCRIPTION

### I. Introduction

This method provides, among other things, systems and methods for analyzing complex biological samples that comprise one or a plurality of different kinds of molecules, e.g., at least any of one, two, three, four, five, six of more biomolecules and other molecular types, such as amino acids or polypeptides (proteins or peptides), carbohydrates (complex carbohydrates or simple carbohydrates such as monosaccharides, disaccharides and trisaccharides) nucleotides or polynucleotides (nucleic acids, RNA, DNA), lipids (fatty acids, complex lipids), salts and metals. Typically, these mixtures will be in the form of cell or tissue homogenates, or isolated and disrupted microvesicles, which may have been treated with enzymes such as proteases, to cleave larger molecules into smaller ones. The system for analysis includes a trap column which is in fluid communication with an analytical column which is in fluid communication with a detector, such as a mass spectrometer. A complex sample is loaded onto the trap column, where debris and non-analyte molecules are removed. The analyte, e.g., peptides, now at least partially separated from other molecules such as salts, fats, nucleic acids and carbohydrates, is moved directly through a fluidic conduit onto the analytical column, where molecules of the same type are sorted based on the property selected by the analytical column. The output of the analytical column is moved directly through a fluidic conduit onto a detector, such as a mass spectrometer, for detection.

This disclosure provides, among other things, systems and methods for analyzing analytes from microparticles. The methods involve, in certain embodiments, the following operations: (a) providing a biological sample; (b) enriching the sample for circulating microparticles; (c) subjecting the enriched sample to particle disruption and proteolytic digestion to produce an analytic sample; (d) loading the analytic sample after (c) onto a trap column that captures polypeptides; (e) washing uncaptured material from the trap column; (f) eluting the captured polypeptides from the trap column and loading the eluted polypeptides onto an analytical column that is in fluid connection with the trap column; (g) separating the polypeptides on the analytical column; and (h) detecting separated polypeptides with a detector.

Also provided herein are systems for sample analysis, e.g., analysis of analytes from microparticles. In certain embodiments, the systems comprise: (a) a trap column; (b) a multi-port valve connected through a first fluid conduit to the trap column; and (c) an analytical column connected through a second fluid conduit to the multi-port valve; wherein the trap column is loaded with a sample enriched for circulating microparticles, wherein the sample has been subjected to particle disruption and proteolytic digestion.

Methods described herein can be faster than traditional methods by providing sharp peaks, as seen in time by intensity plots, that allow more samples in the same amount of time.

Methods described herein significantly improve the sensitivity of detection assays, particularly by mass spectrometry. Sensitivity can be improved by, for example, at least any of two times, five times or ten times. This can be manifested by the ability to detect an analyte at a lower detection limit (that is, at reduced concentration in the same sample). It also can be manifested in the ability to load lower amounts of the same sample onto the column to detect an analyte in the column. For example, the amount of sample loaded onto a system comprising an analytical column alone compared to a system comprising a trap column coupled to an analytical column, can be reduced from about 100 micrograms to about 200 micrograms to about 1 microgram to about 5 micrograms. The methods described herein also significantly increase sample throughput, that is, the time between loading a sample and generating an output is reduced. Throughput can be increased by at least any of 25%, 50%, 100%, 200% or 500%. Without wishing to be limited by theory, cleaning a sample first on a trap column may allow fasting binding and elution from the analytical column.

### II. Biological Samples

A sample for use in the methods of the present disclosure is a biological sample obtained from a subject. As used herein, the term “sample” refers to a composition comprising an analyte. A sample can be a raw sample, in which the analyte is mixed with other materials in its native form (e.g., a source material), a fractionated sample, in which an analyte is at least partially enriched, or a purified sample in which the analyte is at least substantially pure. As used herein, the term “biological sample” refers to a sample comprising biological material including, e.g., polypeptides, polynucleotides, polysaccharides, lipids and higher order levels of these materials such as, microparticles, cells, tissues or organs.

The subject can be, for example, a mammal, a primate, or a human. In certain embodiments, the sample is blood, saliva, tears, sweat, nasal secretions, urine, amniotic fluid or cervicovaginal fluid. In additional embodiments, the sample is a blood sample, which in certain embodiments is serum or plasma. In particular, the biological sample can comprise microparticles such as exosomes and can be derived from blood or a fraction thereof. In further embodiments, the sample is a tissue sample, e.g., a biopsy or a buccal swab. In some embodiments, the sample has been stored frozen (e.g., −20° C. or −80° C.).

A. Subjects

In some embodiments, the subject is a pregnant human. In some embodiments, the pregnant human subject is in the first trimester (e.g., weeks 1-12 of gestation), second trimester (e.g., weeks 13-28 of gestation) or third trimester of pregnancy (e.g., weeks 29-37 of gestation). In some embodiments, the pregnant human subject is in early pregnancy (e.g., from 8, 9, 10, 11, 12, 13, 14, 15, 16, 17, 18, 19 or 20, but earlier than 21 weeks of gestation; from 20, 19, 18, 17, 16, 15, 14, 13, 12, 11, 10 or 9, but later than 8 weeks of gestation). In some embodiments, the pregnant human subject is in mid-pregnancy (e.g., from 21, 22, 23, 24, 25, 26, 27, 28, 29 or 30, but earlier than 31 weeks of gestation; from 30, 29, 28, 27, 26, 25, 24, 23, 22 or 21, but later than 20 weeks of gestation). In some embodiments, the pregnant human subject is in late pregnancy (e.g., from 31, 32, 33, 34, 35, 36 or 37, but earlier than 38 weeks of gestation; from 37, 36, 35, 34, 33, 32 or 31, but later than 30 weeks of gestation). In some embodiments, the pregnant human subject is in less than 17 weeks, less than 16 weeks, less than 15 weeks, less than 14 weeks or less than 13 weeks of gestation; from 20, 19, 18, 17, 16, 15, 14, 13, 12, 11, 10 or 9, but later than 8 weeks of gestation). In some embodiments, the pregnant human subject is in about 8-12 weeks of gestation. In some embodiments, the pregnant human subject is in about 18-24 weeks of gestation. In an exemplary embodiment, the pregnant human subject is at 10-12 weeks of gestation. In some embodiments, the pregnant human subject is in about 22-24 weeks of gestation. The stage of pregnancy can be calculated from the first day of the last normal menstrual period of the pregnant subject.

In some embodiments, the pregnant human subject is primigravida. In other embodiments, the pregnant subject multigravida. In some embodiments, the pregnant subject may have had at least one prior spontaneous preterm birth (e.g., birth prior to week 38 of gestation). In some embodiments, the pregnant human subject is asymptomatic. In some embodiments, the subject may have a risk factor of preterm birth such as a history of pre-gestational hypertension, diabetes mellitus, kidney disease, known thrombophilias and/or other significant preexisting medical condition (e.g., short cervical length).

In some embodiments, the sample is obtained from a subject who has, or is suspected of having, or is being screened for, a cancer or a tumor. The term “cancer” can refer to leukemias, carcinomas, sarcomas, adenocarcinomas, lymphomas, solid and lymphoid cancers, etc. Examples of different types of cancer include, but are not limited to, lung cancer (e.g., non-small cell lung cancer or NSCLC), breast cancer, prostate cancer, colorectal cancer, bladder cancer, ovarian cancer, leukemia, liver cancer (i.e., hepatocarcinoma), renal cancer (i.e., renal cell carcinoma), thyroid cancer, pancreatic cancer, uterine cancer, cervical cancer, testicular cancer, esophageal cancer, stomach (gastric) cancer, cancer of the central nervous system, skin cancer, glioblastoma and melanoma.

B. Microparticles

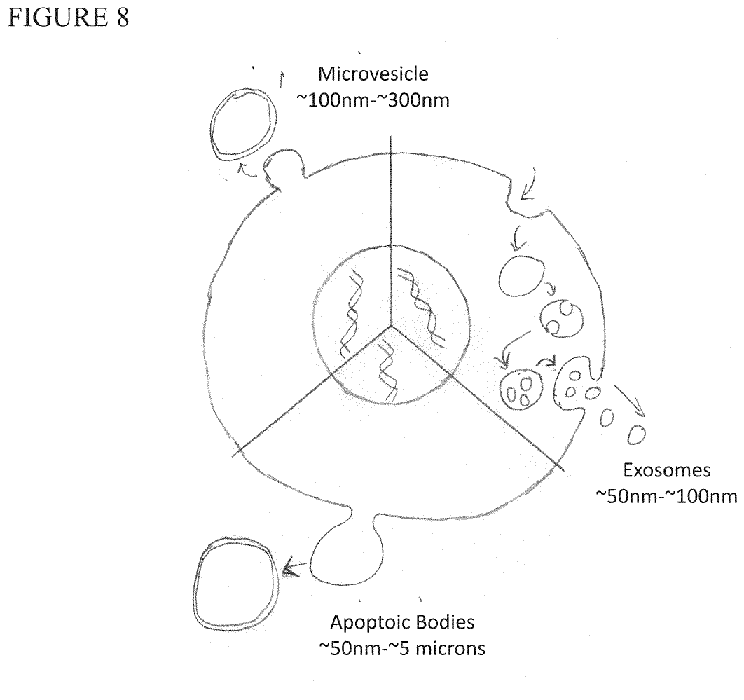

FIG. 8 shows a cell producing three kinds of microparticles, microvesicles, apoptotic bodies and exosomes. As used herein, the term “microparticle” refers to an extracellular microvesicle or lipid raft protein aggregate having a hydrodynamic diameter of from about 50 nm to about 5000 nm. As such the term microparticle encompasses exosomes (about 50 nm to about 100 nm), microvesicles (about 100 nm to about 300 nm), ectosomes (about 50 nm to about 1000 nm), apoptotic bodies (about 50 nm to about 5000 nm) and lipid protein aggregates of the same dimensions. As used herein, the term “about” as used in reference to a value refers to 90 to 110% of that value. For instance, a diameter of about 1000 nm is a diameter within the range of 900 nm to 1100 nm.

Increasingly, microparticles are recognized as important means of intercellular communication in physiologic, pathophysiologic and apoptotic circumstances. While the contents of different types of microparticles vary with cell type, they can include nuclear, cytosolic and membrane proteins, as well as lipids, messenger RNAs and micro RNAs, and small molecules derived from metabolism. Information regarding the state of the cell type of origin can be derived from an examination of microparticle contents. Thus, microparticles represent a unique and convenient window into real-time activities of cells, tissues and organs that may otherwise be difficult to sample.

As used herein, the term “polypeptide” refers to a polymer comprising amino acids attached through peptide bonds. The term embraces “proteins”, which can include large polypeptides having, in addition to a primary structure (an amino acid sequence), a secondary structure (a pattern of hydrogen bonds that determine a general three-dimensional form of local segments of a polypeptide, e.g., alpha-helix or beta-sheet), a tertiary structure (three-dimensional shape) and/or a quaternary structure (a number and arrangement of multiple protein molecules in a multi-subunit complex). Proteins may be modified by, for example, glycosylation, lipidation, phosphorylation, etc. The term also embraces “peptides”, e.g., short polypeptides typically having no more than about 50 amino acids.

“Fragments” of a protein include polypeptides that are shorter in length than the full length or mature protein of interest. A fragment may be as short as 4 amino acids, but is preferably longer (e.g., up to any of 5, 6, 7, 8, 9, 10, 12, 15, 20, 25, 30, 35, 40, 50, 60, 70, 80, 90, or 100 amino acids or any integral value therebetween). A fragment can be any portion of the protein that is shorter than the full-length protein, and does not necessarily have to possess one of the ends of the full-length protein. The average trypsin-generated fragment is about 6 to about 14 amino acids long. In addition to the number of amino acid residues, protein fragments can also be characterized by their molecular weight. Since the average molecular weight of an amino acid is 110 Da; a 10-mer peptide will have an approximate molecular weight of 1,100 Da (1.1 kDA); a 50-mer will have an approximate molecular weight of 5,500 Da (5.5 kDA), etc.

A microparticle-associated protein refers to a protein or fragment thereof (e.g., polypeptide or peptide) that is detectable in a microparticle-enriched sample, e.g., from a mammalian (e.g., human) subject. As such, a microparticle-associated protein is not restricted to proteins or fragments thereof that are physically associated with microparticles at the time of detection; the proteins or fragments may be incorporated between microparticles, or the proteins or fragments may have been associated with the microparticle at some earlier time prior to detection. In certain embodiments, a microparticle-associated protein resides within a microparticle and can be released by disrupting the microparticle, for example, by contacting the microparticle with chaotropic agents, reducing agents, alkylating agents and/or proteases.

Numerous microparticle-associated proteins have been determined to be altered in samples from subjects with pathological conditions (e.g., cancer, preterm birth), as compared to samples from subjects not having the condition, and can therefore be used as biomarkers for the pathological condition. As used herein, “alteration of a microparticle-associated protein” refers to a change in the amount (i.e., level) or composition of the microparticle-associated protein. Changes in composition of a protein can include changes in amino acid sequence, changes in glycosylation, changes in phosphorylation, changes in ubiquitination, and/or changes in lipidation.

III. Methods for Enrichment of Circulating Microparticles (CMPs)

CMPs, e.g. exosomes, from plasma samples, can be enriched by size exclusion chromatography (SEC) and isocratically eluted using water (RNAse free, DNAse free, distilled water) or PBS. For example, PD-10 columns (GE Healthcare Life Sciences) are packed with 10 mL of 2% agarose bead (standard pore size 50-150 μm from ABT, Miami, Fla.), washed (e.g., with water or PBS) and stored at 4° C. for a minimum of 24 hrs and no longer than three days prior to use. On the day of use columns are washed (e.g., with water or PBS) and 1 mL of thawed neat plasma sample (i.e., the plasma sample is not filtered, diluted or treated prior to chromatography) is applied to the column. Circulating microparticles are captured in the column void volume, partially resolved from the peak of high abundance proteins. Ezrin et al. (2015) Am. J. Perinatol. 32:605-614.

The methods described herein for microparticle (e.g., exosome) enrichment have the further advantage of depleting the microparticle sample of high-abundance non-analyte serum proteins such as albumin, haptoglobin and antibodies. In addition, by eluting microparticles from the column with water or a low ionic strength buffer, the enriched microparticles are obtained in a low ionic strength solution, minimizing changes in column chemistry (e.g., pH, ionic strength) that occur when high ionic strength samples, such as are obtained without purification, are applied to the trap column and fractionated on the analytical column.

IV. Particle Disruption and Proteolysis

To release microparticle (e.g., exosome)-associated proteins and convert said proteins to smaller peptides amenable to analysis by MS, exosomes are treated such that their contents are released, and proteins are digested. For example, exosomes (optionally enriched by, e.g., size-exclusion chromatography) can be disrupted and proteins converted to peptides by contacting exosomes with chaotropic agents, denaturing agents, reducing agents, alkylating agents and/or proteases. Chaotropic agents and denaturing agents destroy the integrity of the particle such that the contents are released. Denaturing agents also unfold proteins, e.g., by breaking hydrogen bonds, thereby making proteins more susceptible to proteolysis. Reducing agents and alkylating agents break disulfide bonds, which also facilitates protein unfolding.

Exemplary chaotropic agents and denaturing agents include urea, guanidinium chloride, sodium perchlorate, lithium perchlorate, lithium acetate and thiourea. Reducing agents are known in the art and include, for example, dithiothreitol (DTT), dithioerythritol (DTE), tris (2-carboxyethyl) phosphine, glutathione and β-mercaptoethanol. Exemplary alkylating agents include; iodoacetamide and iodoacetate, which bind to the thiol group of cysteine residues, thereby breaking disulfide bonds. Other disulfide bond-breaking agents, as are known in the art, can also be used in place of, or together with, an alkylating agent.

Proteolytic enzymes and proteolytic agents are well-known in the art. Exemplary proteolytic enzymes include aminopeptidases, carboxypeptidases, trypsin, chymotrypsin, bromelian, papain, pronase and Proteinase K. The chemical agent cyanogen bromide (CnBr) can also be used as a proteolytic agent.

As used herein, the term “microparticle preparation” or “exosome preparation” refers to a sample in which microparticles or exosomes are disrupted and polypeptides are fragmented, e.g., into peptides.

### V. System and Methods

Systems as disclosed herein can include a trap column fluidically connected to a valve having a first port fluidically connected with a container and a second port fluidically connected to an analytical column. In certain embodiments, the analytical column is further fluidically connected to a detector. As used herein, a first apparatus is fluidically connected to a second apparatus if the first and second apparatus are connected by a conduit adapted for directing the flow of fluid or liquid. The conduit can be, for example, a tube or line having a lumen through which fluid can travel. A first apparatus is in fluid communication with a second apparatus when they are connected by a continuous stream of fluid.

Methods of fractionating microsome or exosome preparations for analyte detection include loading the microsome or exosome prep onto a trap column to capture peptides, eluting waste materials from the trap column, e.g., by sending them through a fluidic connection to a valve to a waste container, and then eluting analytes through a fluidic connection to an analytical column. Analytes loaded on the analytical column can be separated on the analytical column and then analyzed, for example, by passing them through a fluidic connection to a detector, such as a mass spectrometer.

Reverse phase or hydrophobic chromatography columns can employ C1-C18 aliphatic hydrocarbons or aromatic hydrocarbon functional groups such as phenyl groups. Normal phase or hydrophilic chromatography columns can employ silicon-oxides (i.e., glass) as adsorbents. Cation exchange chromatography columns can employ sulfate anions (i.e., SO₃⁻), carboxylate anions (i.e., COO⁻), or phosphate anions (OPO₃⁻). Anion exchange chromatography columns can employ secondary, tertiary or quaternary amines, such as ammonium ions. Immobilized dye chromatography columns can employ dyes such as CIBACHRON™ blue. Metal ion chromatography columns can employ metal chelators such as copper, nickel, cobalt, zinc, and iron. Other selective resins include triethylammonium, thiol, aminophosphonic, iminodiacetic, methyl glucamine, bis-picolylamine, and thiourea. Biospecific chromatography columns can employ molecules (such as antibodies, aptamers, proteins receptors or nucleic acids with specific nucleotide sequences) that specifically bind target molecules.

A. Tandem Column System

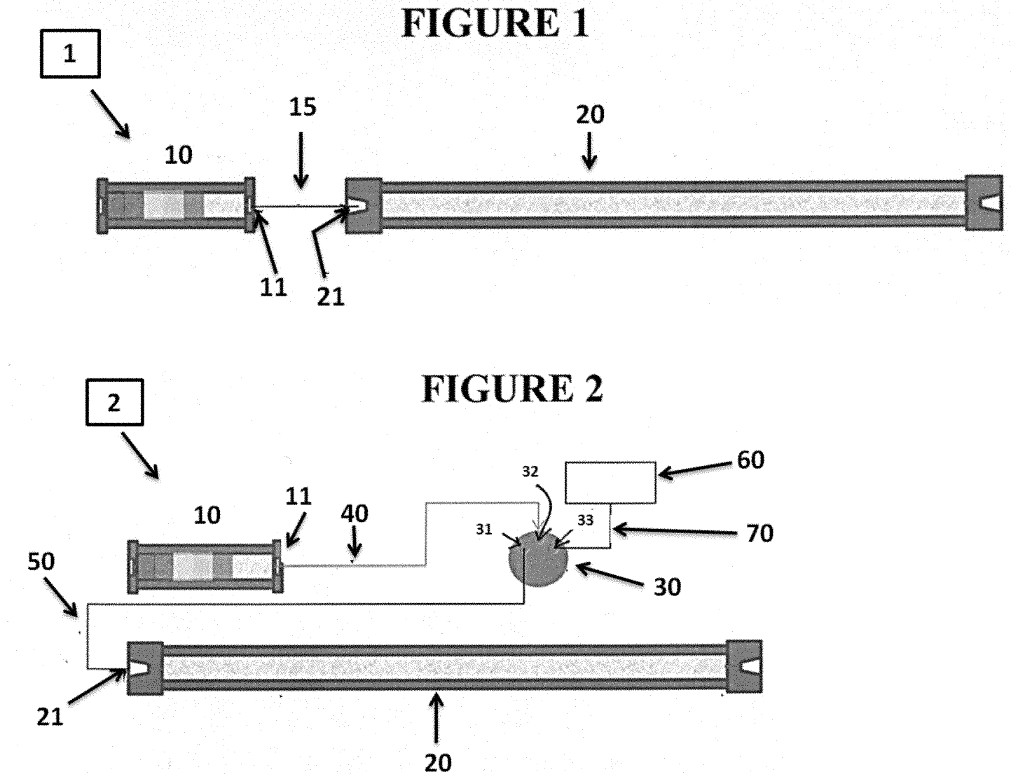

In certain embodiments, the present disclosure provides a tandem paired column system for preparing samples for analysis, e.g., by MS. In an exemplary embodiment as shown in FIG. 1, the system 1 comprises trap column 10 and analytical column 20 in fluid connection with each other, e.g., via fluid conduit 15.

In certain embodiments, as shown in FIG. 2, trap column 10 and analytical column 20 are fluidly connected via multiport valve 30. Multiport valve 30 can contain a number of ports (e.g., 31, 32 and 33) allowing access to, and egress from, the valve. In additional embodiments, trap column 10 is connected to valve 30 via first fluid conduit 40 that connects to intake port 32 on valve 30. In further embodiments, valve 30 is connected to analytical column 20 via second fluid conduit 50 that is attached to outlet port 31 on valve 30. In additional embodiments, valve 30 flows to waste or is connected to waste container 60 through third fluid conduit 70 that attaches to outlet port 33 on valve 30.

In additional embodiments, as shown in FIG. 3, analytical column 20 is connected to detector 80 through fourth fluid conduit 90.

In additional embodiments, as shown in FIG. 4, pump 100 is connected to trap column 10 through fifth fluid conduit 110.

In one embodiment, the system comprises, in upstream to downstream order, pump 100 connected to fifth fluid conduit 110, which is in turn connected to trap column 10, which in turn is connected to first fluid conduit 40, which in turn is connected to multi-port valve 30 via intake port 32, said valve 30 being in turn connected to both second fluid conduit 50 via outlet port 31 and third fluid conduit 70 via outlet port 33. Third fluid conduit 70 may optionally be connected with, or lead into, waste container 60. Second fluid conduit 50 is connected, in turn to analytical column 20 which is connected, in turn, to fourth fluid conduit 90 which is connected, in turn, to detector 80.

B. Trap Column

Any chromatographic resin known in the art, capable of retaining proteins, can be used as a trap column. Trap columns are typically short columns containing high-capacity, low-efficiency resins. They include, for example, anion exchange, cation exchange, size exclusion, reverse phase and polystyrene-divinylbenzene resins can be used. An HPLC column can also be used. Exemplary HPLC resins include C18 and C6.

Trap columns are commercially available from, e.g., ThermoFisher Scientific (Waltham, Mass.). These include, for example, the Dionex™ IonPac™ and OmniPac™ reversed phased columns, as well as ion exchange columns. They also include reversed phase columns from Waters™.

In certain embodiments, the microparticle preparation is loaded onto the trap column in a low salt condition. In certain embodiments, the microparticle preparation is loaded onto the trap column in a “no-salt” condition; e.g., in distilled water after eluting microparticles from a SEC column using distilled water (see above).

C. Analytical Column

Any chromatographic resin known in the art, capable of resolving peptides, can be used as an analytical column. For example, anion exchange, cation exchange, size exclusion and reverse phase, and polystyrene-divinlybenzene resins can be used. An HPLC column can also be used. Exemplary HPLC resins include C18 and C6. In certain embodiments, an analytical column is a 25 cm C18 column. In contrast to current “nano-flow” rates of 10-1000 nl/min; the “normo-flow” rates used in the methods disclosed herein range from 0.25-0.5 ml/min, allowing more rapid identification of analytes.

D. Detector

The present disclosure provides, in certain aspects, methods and compositions for determining a quantitative measure of at least one analyte (e.g., microparticle-associated protein) in a sample. Quantitative measures include, without limitation, presence or absence, absolute or relative amounts or concentrations, absolute or relative increases or decreases and discrete or continuous ranges (e.g., a number, a degree, a level, a threshold, a quantile or a bucket). In some embodiments, the quantitative measure can be an absolute value, a ratio, an average, a median, or a range of numbers.

1. Mass Spectrometer

In some embodiments, detecting the level (e.g., including detecting the presence) of a microparticle-associated protein is accomplished using a liquid chromatography/mass spectrometry (LCMS)-based proteomic analysis. In an exemplary embodiment the method involves subjecting a sample to size exclusion chromatography and collecting the high molecular weight fraction (e.g., by size-exclusion chromatography) to obtain a microparticle-enriched sample. The microparticle-enriched sample is then disrupted (using, for example, chaotropic agents, denaturing agents, reducing agents and/or alkylating agents) and the released contents subjected to proteolysis. The disrupted exosome preparation, containing a plurality of peptides, is then processed using the tandem column system described herein prior to peptide analysis by mass spectrometry, to provide a proteomic profile of the sample. The methods disclosed herein avoid the necessity of protein concentration/purification, buffer exchange and liquid chromatography steps associated with previous methods.

Proteins in a sample can be detected by mass spectrometry. Mass spectrometers typically include an ion source to ionize analytes, and one or more mass analyzers to determine mass. Mass analyzers can be used together in tandem mass spectrometers. Ionization methods include, among others, electrospray or laser desorption ionization. Mass analyzers include quadrupoles, ion traps, time-of-flight instruments and magnetic or electric sector instruments. In certain embodiments, the mass spectrometer is a tandem mass spectrometer (e.g., “MS-MS”) that uses a first mass analyzer to select ions of a certain mass and a second mass analyzer to analyze the selected ions. One example of a tandem mass spectrometer is a triple quadrupole instrument, the first and third quadrupoles act as mass filters, and an intermediate quadrupole functions as a collision cell. Mass spectrometry also can be coupled with up-stream separation techniques, such as liquid chromatography or gas chromatography. So, for example, liquid chromatography coupled with tandem mass spectrometry can be referred to as “LC-MS-MS”.

Mass spectrometers useful for the analyses described herein include, without limitation, Altis™ quadrupole, Quantis™ quadrupole, Quantiva™ or Fortis™ triple quadrupole from ThermoFisher Scientific, and the QSight™ Triple Quad LC/MS/MS from Perkin Elmer.

Selected reaction monitoring is a mass spectrometry method in which a first mass analyzer selects a polypeptide of interest (precursor), a collision cell fragments the polypeptide into product fragments and one or more of the fragments is detected in a second mass analyzer. The precursor and product ion pair is called a SRM “transition”. The method is typically performed in a triple quadrupole instrument. When multiple fragments of a polypeptide are analyzed, the method is referred to as Multiple Reaction Monitoring Mass Spectrometry (“MRM-MS”). Typically, protein samples are digested with a proteolytic enzyme, such as trypsin, to produce peptide fragments. Heavy isotope labeled analogues of certain of these peptides are synthesized as standards. These standards are referred to as Stable Isotopic Standards or “SIS”. SIS peptides are mixed with a protease-treated sample. The mixture is subjected to triple quadrupole mass spectrometry. Peptides corresponding to the SIS standards are detected with high accuracy. Peptides can be synthesized to order, or can be available as commercial kits from vendors such as, for example, e.g., ThermoFisher (Waltham, Mass.) or Biognosys (Zurich, Switzerland).

Accordingly, detection of a protein target by MRM-MS involves detection of one or more peptide fragments of the protein, typically through detection of a stable isotope standard peptide against which the peptide fragment is compared.

Generally, any mass spectrometric (MS) technique that can provide precise information on the mass of peptides, and preferably also on fragmentation and/or (partial) amino acid sequence of selected peptides (e.g., in tandem mass spectrometry, MS/MS; or in post source decay, TOF MS), can be used in the methods and compositions disclosed herein. Suitable peptide MS and MS/MS techniques and systems are known in the art (see, e.g., Methods in Molecular Biology, vol. 146: “Mass Spectrometry of Proteins and Peptides”, by Chapman, ed., Humana Press 2000; Kassel & Biemann (1990) Anal. Chem. 62:1691-1695; Methods Enzymol 193: 455-79; or Methods in Enzymology, vol. 402: “Biological Mass Spectrometry”, by Burlingame, ed., Academic Press 2005) and can be used in practicing the methods disclosed herein. Accordingly, in some embodiments, the disclosed methods comprise performing quantitative MS to measure one or more peptides. Such quantitative methods can be performed in an automated (Villanueva, et al., Nature Protocols (2006) 1(2):880-891) or semi-automated format. In particular embodiments, MS can be operably linked to a liquid chromatography device (LC-MS/MS or LC-MS) or gas chromatography device (GC-MS or GC-MS/MS).

As used herein, the terms “multiple reaction monitoring (MRM)” or “selected reaction monitoring (SRM)” refer to a MS-based quantification method that is particularly useful for quantifying analytes that are in low abundance. In an SRM experiment, a predefined precursor ion and one or more of its fragments are selected by the two mass filters of a triple quadrupole instrument and monitored over time for precise quantification. Multiple SRM precursor and fragment ion pairs can be measured within the same experiment on the chromatographic time scale by rapidly toggling between the different precursor/fragment pairs to perform an MRM experiment. A series of transitions (precursor/fragment ion pairs) in combination with the retention time of the targeted analyte (e.g., peptide or small molecule such as chemical entity, steroid, hormone) can constitute a definitive assay. A large number of analytes can be quantified during a single LC-MS experiment. The term “scheduled,” or “dynamic” in reference to MRM or SRM, refers to a variation of the assay wherein the transitions for a particular analyte are only acquired in a time window around the expected retention time, significantly increasing the number of analytes that can be detected and quantified in a single LC-MS experiment and contributing to the selectivity of the test, as retention time is a property dependent on the physical nature of the analyte. A single analyte can also be monitored with more than one transition. Finally, the assay can include standards that correspond to the analytes of interest (e.g., peptides having the same amino acid sequence as that of analyte peptides), but differ by the inclusion of stable isotopes. Stable isotopic standards (SIS) can be incorporated into the assay at precise levels and used to quantify the corresponding unknown analyte. Additional levels of specificity are contributed by the co-elution of the unknown analyte and its corresponding SIS, and by the properties of their transitions (e.g., the similarity in the ratio of the level of two transitions of the analyte and the ratio of the two transitions of its corresponding SIS).

Mass spectrometry assays, instruments and systems suitable for biomarker peptide analysis can include, without limitation, matrix-assisted laser desorption/ionization time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)n (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS)n; ion mobility spectrometry (IMS); inductively coupled plasma mass spectrometry (ICP-MS) atmospheric pressure photoionization mass spectrometry (APPI-MS); APPI-MS/MS; and APPI-(MS)n. Peptide ion fragmentation in tandem MS (MS/MS) arrangements can be achieved using techniques known in the art, such as, e.g., collision induced dissociation (CID). As described herein, detection and quantification of biomarkers by mass spectrometry can involve multiple reaction monitoring (MRM), such as described, inter alia, by Kuhn et al. (2004) Proteomics 4:1175-1186. Scheduled multiple-reaction-monitoring (Scheduled MRM) mode acquisition during LC-MS/MS analysis enhances the sensitivity and accuracy of peptide quantitation. Anderson and Hunter (2006) Mol. Cell. Proteomics 5(4):573-588. Mass spectrometry-based assays can be advantageously combined with upstream peptide or protein separation or fractionation methods, such as, for example, with the tandem column system described herein.

### VI. Diagnostic Methods

Proteomic analyses, as disclosed herein, allow the identification of proteins, in biological samples, that are markers for pathological conditions. Protein and peptide markers for pathological conditions are known in the art. For example, disorders of pregnancy, such as spontaneous pre-term birth, can be predicted by the presence of certain proteins in a biological sample from a pregnant woman. See, for example, WO 2017/096405. Cancer markers are also known. See, for example, Zhu et al. (2011) Oncogene 30:457-470; and Wang et al. (2017) Proc. Natl. Acad. Sci. USA 114:13519-13524.

### EXAMPLES

Frozen maternal plasma samples are used. Samples may be transported on dry ice and stored at −80° C.

Microparticles are enriched by Size Exclusion Chromatography (SEC) and isocratically eluted using water (RNAse free, DNAse free, distilled water). The size exclusion chromatography procedure is performed using columns manufactured by AmericanBio (Natick, Mass.). Briefly, these columns are packed with 10 mL of 2% Sepharose 2B (pore size 60-200 um) from GE Healthcare Life Sciences (Marlborough, Mass.). The columns are stored under refrigerated conditions. Prior to using the columns for exosome isolation, they are allowed to equilibrate to room temperature. Following equilibration to room temperature, the columns are washed with RNAse free, DNAse free, distilled water. A minimum of 0.5 mL of thawed neat plasma is applied to the column. Microparticles are eluted with RNAse free, DNAse free, distilled water.

The circulating microparticles are captured in the column void volume, partially resolved from the high abundant protein peak. One aliquot of the pooled CMP column fraction from each clinical specimen, containing 200 ug of total protein (determined by BCA) is used for further analysis.

Exosomes are disrupted by adjusting the sample to 8 Molar urea and proteins are digested with Trypsin Gold, Mass Spectrometry Grade (Promega, 100 μg). Isotopically labeled peptides for ITIH4 and/or IC1 are added to the sample for MRM analysis. The sample is divided into first and second aliquots.

A first aliquot is loaded onto a ThermoFisher Vanquish Flex UHPLC C18 analytical column and analyzed on a ThermoFisher TSQ Quantiva Triple Quadrupole LC-MS/MS system. A second aliquot is loaded onto a polystyrene-divinlybenzene trap column. Unbound material is eluted to waste. Remaining material is passed to a ThermoFisher Vanquish Flex UHPLC C18 analytical column before being analyzed on a T ThermoFisher TSQ Quantiva Triple Quadrupole LC-MS/MS system.

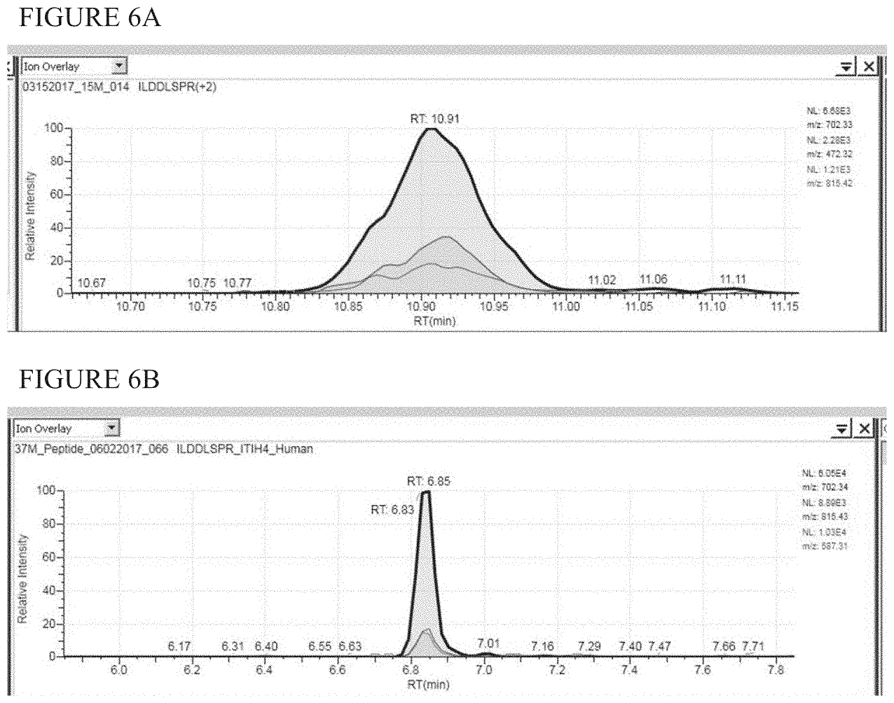

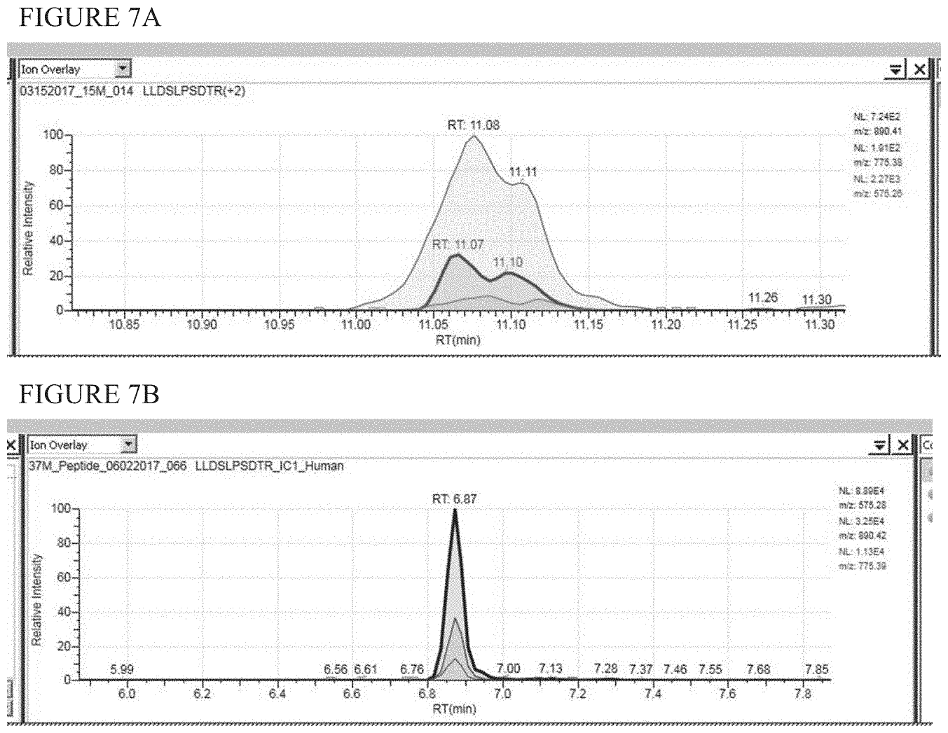

Exemplary results for the type of protocol above for ITIH4 analysis are shown in FIGS. 6A and 6B. Three overlapping fragments of an ITIH4 peptide having the sequence ILDDLSPR (SEQ ID NO: 1) are shown in the time dimension. Results for IC1 analysis are shown in FIGS. 7A and 7B. Three fragments of an IC1 peptide having the sequence LLDSLPSDTR (SEQ ID NO: 2) are shown in the time dimension. Samples loaded directly onto the analytical column produce low intensity, noisy peaks, while samples first purified on a trap column before being loaded onto the analytical column show much more abundant and sharper peaks with higher intensity.

The Table 1 displays the reproducibility for IC1 analysis whereby the samples are directly loaded onto the analytical column—this data corresponds to the experiments run in FIG. 7A. The low intensity, noisy peaks are correlated with a high variability, with CVs in the 40% to 50% range; this would result in unreliable measurements, and an inability to discriminate between two samples unless their differences in values were very large (e.g. a sample would have to have a value of greater than 1.1 ng/mL vs. a sample of 0.55 ng/mL to be reliably quantified as different in concentration). The mean values are lower compared to the trap method, probably because of loss of sample and weak detection by the spectrometer. The CVs reported here represent the reproducibility of the platform where the sample is taken all the way through the entire process, from enrichment of exosomes on the columns through measurement of the target peptides on the mass spectrometer.

TABLE 1
Representative reproducibility of exosome-mass spectrometry
platform without the in-line trap for the IC1 peptide.
IC1 Reproducibility in ng/mL

| | |
|---|---|
| Mean | 0.55 |
| SD | 0.29 |
| % CV | 52.39 |

The Table displays the reproducibility for IC1 analysis whereby the samples are first purified on the trap before being loaded onto the analytical column—this data corresponds to the experiments run in FIG. 7B. The significantly higher intensity, sharper peaks are correlated with a low variability, with CVs in the 4% range; this would result in very reliable measurements, comparing very favorably compared to ELISAs and other antibody-based technologies, and an ability to discriminate between two samples that have mean values close to each other (e.g. a value of 12.2 ng/mL could reliably be distinguished from a sample with a value of 11.3 ng/mL). The mean values are higher with the trap method, probably because of little loss of sample during preparation and the sensitive detection of the peptide by the spectrometer. The CVs reported here represent the reproducibility of the platform where the sample is taken all the way through the entire process, from enrichment of exosomes on the columns through measurement of the target peptides on the mass spectrometer. The response ratio is a measure of the ratio between the standard peptide and the endogenous peptide. This is used for quantification along with a Standard Curve. The response ratio has great reproducibility indicating a reliable detection of the heavy (standard) and light (endogenous) peptides and their relative concentrations; this is another indicator of the high performance of the system.

TABLE 2
Representative reproducibility of exosome-mass spectrometry
platform with the in-line trap in place for the IC1 peptide.

| | IC1 Lab Values | |
|---|---|---|
| | Overall in ng/mL | Response Ratio |
| Mean | 11.312 | 0.006 |
| SD | 0.474 | 0.001 |
| % CV | 4.1942 | 10.8202 |

The technician to technician variability is low—the values below make up the overall average for reproducibility in Table 2 above, which demonstrates an excellent intra-operator repeatability (variation in measurements taken by a single instrument or person under the same conditions) and overall reproducibility.

TABLE 3
Reproducibility of exosome platform for different technicians.

| | IC1 Technician 1 | IC1 Technician 2 in |
|---|---|---|
| | in ng/mL | ng/mL |
| Mean | 11.43833333 | 11.18566667 |
| SD | 0.368485187 | 0.615704745 |
| % CV | 3.221493697 | 5.504408129 |

Unless otherwise stated herein, the methods and compositions disclosed herein utilize standard procedures in the fields of biological samples (e.g., blood draw, biopsy, buccal swab) and peptide processing, peptide analysis and mass spectrometry.

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The phrase “at least one” includes “one”, “one or more”, “one or a plurality” and “a plurality”. The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”. The term “consisting essentially of” refers to the inclusion of recited elements and other elements that do not materially affect the basic and novel characteristics of a claimed combination.

It should be understood that the description and the drawings are not intended to limit the invention to the particular form disclosed, but to the contrary, the intention is to cover all modifications, equivalents, and alternatives falling within the spirit and scope of the present invention as defined by the appended claims. Further modifications and alternative embodiments of various aspects of the invention will be apparent to those skilled in the art in view of this description. Accordingly, this description and the drawings are to be construed as illustrative only and are for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims. Headings used herein are for organizational purposes only and are not meant to be used to limit the scope of the description.

1. A method comprising:
(a) providing a biological sample;
(b) enriching the sample for circulating microparticles;
(c) subjecting the enriched sample to particle disruption and proteolytic digestion to produce an analytic sample;
(d) loading the analytic sample onto a trap column that captures polypeptides;
(e) washing uncaptured material from the trap column;
(f) eluting the captured polypeptides from the trap column and loading the eluted polypeptides onto an analytical column that is in fluid connection with the trap column;
(g) separating the polypeptides on the analytical column; and
(h) detecting separated polypeptides with a detector.

2. The method of claim 1, wherein the biological sample comprises blood, serum, plasma, saliva, cerebrospinal fluid, amniotic fluid or urine.

3. The method of claim 1, wherein the biological sample is provided from a subject having a condition selected from pregnancy, cancer, spontaneous pre-term birth, pre-eclampsia or diabetes.

4. The method of claim 1, wherein, the biological sample is enriched for microparticles by one or more of size exclusion chromatography, ultrafiltration or reverse-phase chromatography.

5. The method of claim 1, wherein the circulating microparticles are exosomes.

6. The method of claim 1, wherein the particle disruption comprises exposure to one or more of urea, a reducing agent (such as, DTT, DTE, mercaptoethanol, tris (2-carboxyethyl) phosphine or glutathione) or iodoacetamide.

7. The method of claim 1, wherein proteolytic digestion comprises exposing the sample to cyanogen bromide (CnBr) or to one or more proteases such as an aminopeptidase, a carboxypeptidase, trypsin, chymotrypsin, bromelian, papain, pronase, and proteinase k.

8. The method of claim 1, comprising adding stable isotope standard peptides for detecting specific protein markers to the analytical sample.

9. The method of claim 1, wherein the trap column performs chromatography selected from reverse phase chromatography, anion exchange chromatography, cation exchange chromatography or size exclusion chromatography or is a polystyrene-divinlybenzene column.

10. The method of claim 1, wherein the trap column comprises a high performance liquid chromatography (HPLC) column.

11. The method of claim 10, wherein the HPLC column comprises a reverse phase material selected from C18 or C6.

12. The method of claim 1, wherein the uncaptured material in step (e) comprises material selected from salts and macromolecules or complexes having a size greater than 5.5 kD or the macromolecule is a polypeptide greater than about 50 amino acids long.

13. The method of claim 1, wherein the analytical column comprises a high-performance liquid chromatography (HPLC) column.

14. The method of claim 13, wherein the HPLC column comprises a reverse phase material selected from C18 or C6.

15. The method of claim 1, wherein the fluid connection between the trap column and the analytical column comprises a first fluid conduit and a second fluid conduit, with a multiport valve disposed therebetween.

16. The method of claim 1, wherein detecting comprises providing separated analytes to the detector through a fluid connection between the analytical column and the detector.

17. The method of claim 1, wherein the detector detects analytes by mass spectrometry.

18. The method of claim 1, wherein the detector comprises a mass spectrometer selected from quadrupole mass spectrometer or time-of-flight mass spectrometer.

19. The method of claim 1, wherein the detector comprises a tandem mass spectrometer.

20. The method of claim 1, wherein the detector comprises a triple quadrupole mass spectrometer.

21. The method of claim 1, wherein the detector comprises a triple quadrupole mass spectrometer and detection comprises multiple reaction monitoring.

22-47. (canceled)
